(19) United States
(12) Patent Application Publication
(10) Pub. No.: US 2012/0115167 A1
(43) Pub. Date: May 10, 2012

(54) METHOD AND APPARATUS FOR ISOLATING A TARGET BIOENTITY FROM A BIOLOGICAL SAMPLE

(75) Inventors: Howard Milne Chandler, Victoria (AU); Michael Bruce Chandler, Victoria (AU)

(73) Assignee: CLINICAL GENOMICS PTY. LTD., North Ryde, New South Wales (AU)

(21) Appl. No.: 13/265,674

(22) PCT Filed: Apr. 22, 2010
(86) PCT No.: PCT/AU2010/000459

§ 371 Date: Dec. 21, 2011

Related U.S. Application Data
(60) Provisional application No. 61/171,532, filed on Apr. 22, 2009.
(60) Provisional application No. 61/182,661, filed on May 29, 2009.
(60) Provisional application No. 61/285,286, filed on Dec. 10, 2009.

(51) Int. Cl. G01N 33/574; B01D 11/02; C12N 13/00; G01N 33/566; C07K 1/36; C07H 1/08

(52) U.S. Cl. 435/7.23; 530/402; 536/25.4; 435/173.9; 436/501; 536/127; 422/261

(57) ABSTRACT

A method for isolating a target bioentity from a biological sample, comprises contacting the biological sample with a magnetically-labelled ligand having selective binding affinity for the target bioentity, or for a determinant on the target bioentity, to form a target bioentity/labelled ligand complex, locating a separation module having a defined fluid flow path in the magnetic field of a magnetic module, the magnetic module comprising an array of at least two magnets in which adjacent magnets in the array are aligned with opposing polarity; passing the biological sample through the separation module to subject the biological sample to the magnetic field while the sample is passing through the defined fluid flow path to magnetically capture the target bioentity/labelled ligand complex by arresting or hindering movement of the complex within the defined fluid flow path; removing the separation module from the magnetic field; and recovering the target bioentity/labelled ligand complex from the fluid flow path.

### REFERENCE TO RELATED APPLICATIONS

This application claims priority to U.S. Provisional Patent Application No. 61/171,532, filed 22 Apr. 2009, U.S. Provisional Patent Application No. 61/182,661, filed 29 May 2009, and U.S. Provisional Patent Application No. 61/285,286, filed 10 Dec. 2009. The contents of the prior applications are hereby incorporated in their entirety.

### FIELD OF THE INVENTION

This invention relates to the isolation of a target bioentity from a biological sample. In one particular embodiment, the invention relates to the isolation and concentration of rare or very rare circulating tumour cells from a blood sample, however the present invention is not limited to this embodiment, and extends more generally to the isolation of a wide range of other target cellular, sub-cellular, and molecular bioentities such as non-tumour cells, including microorganisms and viruses, membrane bound vesicles, sub-cellular proteins, carbohydrates and nucleic acids, contaminants and toxins, and even elements such as rare earth elements.

### BACKGROUND OF THE INVENTION

Broadly speaking, the present invention relates to the isolation of cellular or molecular target bioentities from complex mixtures which make up biological samples, particularly blood, plasma and serum samples.

As indicated above, in one particular embodiment the present invention provides a method for the isolation and concentration from a blood sample of rare or very rare tumour cells or their sub-cellular components which have shed, leaked or migrated into the circulatory system from cancerous or precancerous tumours. Early detection of these rare and very rare tumour cells or their sub-cellular components is of utmost importance and has a very significant effect on the prognosis and outcome of malignant disease in humans and other animals. Recent developments in cancer research have shown that, although they might be rare or even very rare, circulating tumour cells can be detected in peripheral blood samples using appropriate biomarkers to assist in diagnosis of cancerous or precancerous conditions in a patient and in monitoring therapeutic response in a cancer patient.

Similarly, isolation of circulating fetal cells in maternal blood samples can be used in prenatal diagnosis, and isolation of antigen-specific lymphocytes can be used in immune monitoring.

The present invention also provides a method for isolation and concentration of circulating stem cells from a blood sample.

Bodey, U.S. Pat. No. 6,008,002, discloses a composition and method for detecting and isolating antigen associated cancer cells. The composition and method utilize an antigen specific, immunomagnetic composition as the detecting and isolating agent. An illustrative immunomagnetic composition comprises avidin or streptavidin conjugated to paramagnetic beads and further conjugated to antigen specific, biotinylated antibody. A fluidic admixture of the antigen associated cancer cells with the immunomagnetic composition in an affinity column disposed in a magnetic field produces a cancer cell immunomagnetic composition conjugate which is deposited onto the inner wall of the column.

Saur et al., U.S. Pat. No. 4,710,472, discloses a magnetic separation device suitable for removing magnetic bead-coated cells from a system comprising a base, a plurality of magnets mounted on the base such that a sample chamber is created for holding sample containers which are to be placed in close proximity to the magnets, and a means for adjusting the position of the magnets with respect to the sample container. A blood sample containing magnetic bead-coated cells is passed through tubing which is mounted in close proximity to the magnets. The magnetic bead-coated cells are attracted by the magnets and retained in the tubing while the nonmagnetic cells pass through the tubing.

Davidson et al., U.S. Pat. No. 6,482,328 discloses a method and apparatus for magnetically separating target particles of a selected type from a sample in order to produce a concentration of the target particles in the sample, or a depletion of the sample with respect to the target particles, by producing a sample mixture of the sample with magnetic particles having a selective affinity to magnetically stain the target particles; producing a flow of a buffer liquid through a tube which includes an inlet connectable to a source of buffer liquid, and an outlet for the buffer liquid; after a flow of the buffer liquid has been produced through the tube, introducing the sample mixture into the buffer liquid flowing through the tube such that the buffer liquid forms a continuous liquid carrier for the sample mixture as both are fed through the tube; and applying a magnetic field across the tube at a magnetizing station therein to cause the magnetically-stained target particles to be separated and retained in the buffer liquid within the tube at the magnetizing station.

### SUMMARY OF THE INVENTION

According to one aspect of the present invention, there is provided a method for isolating a target bioentity from a biological sample, which comprises the steps of:

(a) contacting said biological sample with a magnetically-labelled ligand having selective binding affinity for the target bioentity, or for a determinant on the target bioentity, to form a target bioentity/labelled ligand complex in said biological sample;

(b) locating a separation module having a defined fluid flow path in the magnetic field of a magnetic module, said magnetic module comprising an array of at least two magnets in which adjacent magnets in the array are aligned with opposing polarity;

(c) passing said biological sample through the separation module to subject the biological sample to the magnetic field while the sample is passing through the defined fluid flow path to magnetically capture the target bioentity/labelled ligand complex by arresting or hindering movement of said complex within the defined fluid flow path;

(d) optionally, and preferably, passing a wash solution through the defined fluid flow path while said complex is magnetically captured within the fluid flow path;

(e) removing the separation module from the magnetic field; and

(f) recovering said target bioentity/labelled ligand complex from the fluid flow path.

In another aspect, the present invention also provides an apparatus for use in isolating a target bioentity from a biological sample which comprises:

(i) a separation module having a defined fluid flow path;

(ii) a magnetic module having a magnetic field, said magnetic module comprising an array of at least two magnets in which adjacent magnets in the array are aligned with opposing magnetic polarity,

wherein the separation module has a first position in which the fluid flow path is located within the magnetic field of the magnetic module and a second position in which the fluid flow path is removed from said magnetic field; and

(iii) a controller comprising means for passing a biological sample, and optionally a wash solution, through the defined fluid flow path provided by the separation module whereby the biological sample is subjected to the magnetic field of the magnetic module while the sample is passing through the defined fluid flow path.

In one aspect, the invention provides a method for isolating a target bioentity from a biological sample, which comprises the steps of:

(a) contacting said biological sample with a magnetically-labelled ligand having selective binding affinity for the target bioentity, or for a determinant on the target bioentity, to form a target bioentity/labelled ligand complex in said biological sample;

(b) locating a separation module having a defined fluid flow path in the magnetic field of a magnetic module, said defined fluid flow path comprising a plurality of tubular elements having a common inlet and a common outlet, and said magnetic module comprising an array of at least two magnets in which adjacent magnets in the array are aligned with opposing polarity;

(c) passing said biological sample through the separation module to subject the biological sample to the magnetic field while the sample is passing through the defined fluid flow path to magnetically capture the target bioentity/labelled ligand complex by arresting or hindering movement of said complex within the defined fluid flow path;

(d) optionally, and preferably, passing a wash solution through the defined fluid flow path while said complex is magnetically captured within the fluid flow path;

(e) removing the separation module from the magnetic field; and

(f) recovering said target bioentity/labelled ligand complex from the fluid flow path.

In this aspect, the present invention also provides an apparatus for use in isolating a target bioentity from a biological sample which comprises:

(i) a separation module having a defined fluid flow path, said defined fluid flow path comprising a plurality of tubular elements having a common inlet and a common outlet;

(ii) a magnetic module having a magnetic field, said magnetic module comprising an array of at least two magnets in which adjacent magnets in the array are aligned with opposing magnetic polarity,

wherein the separation module has a first position in which the fluid flow path is located within the magnetic field of the magnetic module and a second position in which the fluid flow path is removed from said magnetic field; and

(iii) a controller comprising means for passing a biological sample, and optionally a wash solution, through the defined fluid flow path provided by the separation module whereby the biological sample is subjected to the magnetic field of the magnetic module while the sample is passing through the defined fluid flow path.

In another aspect, the method of the present invention may include the steps of:

(i) collecting said biological sample at a first or collection point;

(ii) adding said sample at said first point to a collection medium comprising said magnetically-labelled ligand;

(iii) subsequently forwarding said collection medium comprising said sample and magnetically-labelled ligand to a second or testing point; and

(iv) completion of said method at said second point.

Preferably, in this aspect of the invention, the first and second points are separated, for example are remote or distant, from one another. By way of example, the first or collection point may be a patient's home, a doctor's surgery or office, or a hospital ward, while the second or testing point may be a central testing facility such as a hospital or commercial test or assay laboratory.

Preferably also, the steps of collection of the sample and addition of the sample to the collection medium on the one hand, and the step of completion of the method on the other hand, are separated in time from one another, in particular by a period of transit time sufficient not only to transport the collection medium from the first point to the second point, but also to enable formation of a target bioentity/labelled ligand complex while the sample is in transit.

### BRIEF DESCRIPTION OF THE DRAWINGS

FIG. 1 is a schematic illustration of the basic elements and fluid circuit used in the examples of the invention herein, showing a sample tube or vessel (1), the Separation module (2), the Magnetic module (3), and a vacuum source and waste reservoir e.g. a 20 or 30 ml syringe (4). Syringe (4) provides a convenient means of drawing the sample from tube (1), through the Separation module (2), which may be placed in, or removed from, the magnetic field of the Magnetic module (3), and then to waste (i.e. the syringe in the illustration). Note that the Separation and Magnetic modules are movable relative to one another so that the Separation module may be introduced to, or removed from, the magnetic field of the Magnetic module.

FIG. 2 is a schematic illustration of a coiled-tube version of the Separation module (22) separated from, but movable into, the magnetic field of the Magnetic module (23), which is mounted on support (26).

FIG. 3 illustrates the Separation and Magnetic modules of an embodiment of the invention suitable for singly or simultaneously running multiple samples past the Magnetic module. As illustrated (FIG. 3a), two Separation module tubes (32) are supported by frame (35), mounted on base (36) so that they may be held within the magnetic field of the Magnetic module (33) (FIG. 3b) or removed from it (FIG. 3c). Addition of more tubes to the Separation module allows more samples to be processed.

FIG. 4 illustrates two embodiments of the invention where the magnets (43) are supported on a frame (46) that is rotatable around the long axis of the tube of the Separation module (42). The magnet pairs may be opposite and parallel to the tube length (FIG. 4a), or in series and parallel to the tube (FIG. 4b).

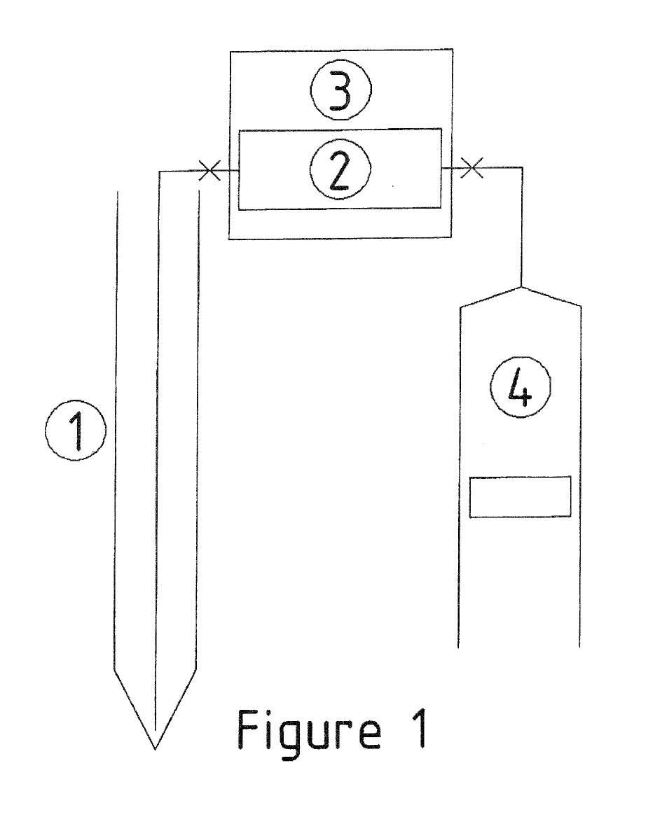

### DETAILED DESCRIPTION OF THE INVENTION

The present invention provides a high throughput device for separation and purification of magnetic particles and of target entities bound or attached to magnetic particles. In particular, the system is designed to be suitable for the rapid and efficient separation of chemical and biological components from fluid samples and to be scalable for industrial and laboratory applications that are not effectively served by current devices. The target bioentities to be separated may include molecules such as agrichemicals, nucleic acids and proteins, sub-cellular components such as exosomes, ribosomes and viruses, and cells, for example circulating tumor cells or circulating stem cells, and microbes, including bacterial pathogens. The fluid samples may include fluids such as water and biological fluids such as blood, plasma, serum, milk, urine and fecal extracts.

Separation of biological and other components from fluid samples by magnetic separation is a well established laboratory procedure and typically involves the following steps:
a) mixing magnetic particles with an affinity for the target entity with a fluid sample suspected of containing the entity in a vessel such as a test tube;
b) allowing time for the particles to bind with the target component;
c) providing a magnet under or around the vessel so that the particles are attracted to the vessel walls;
d) removing the fluid;
e) re-suspending the particles with a wash liquid and repeating steps c) & d) several times to remove inadvertently captured extraneous materials;
f) re-suspending the purified particles, with their specifically captured entity in a suitable buffer or stabilizing fluid.

The present invention improves the speed and efficiency of magnetic separation and purification relative to the established methods by flowing the fluid samples containing magnetic particles past a magnetic module for magnetic arrest of the particles.

In its broadest aspect, the present invention provides a method for isolating a target bioentity from a biological sample, which comprises the steps of

(a) contacting said biological sample with a magnetically-labelled ligand having selective binding affinity for the target bioentity, or for a determinant on the target bioentity, to form a target bioentity/labelled ligand complex in said biological sample;

(b) locating a separation module having a defined fluid flow path in the magnetic field of a magnetic module, said magnetic module comprising an array of at least two magnets in which adjacent magnets in the array are aligned with opposing polarity;

(c) passing said biological sample through the separation module to subject the biological sample to the magnetic field while the sample is passing through the defined fluid flow path to magnetically capture the target bioentity/labelled ligand complex by arresting or hindering movement of said complex within the defined fluid flow path;

(d) optionally, and preferably, passing a wash solution through the defined fluid flow path while said complex is magnetically captured within the fluid flow path;

(e) removing the separation module from the magnetic field; and

(f) recovering said target bioentity/labelled ligand complex from the fluid flow path.

In one aspect, the invention provides a method for isolating a target bioentity from a biological sample, which comprises the steps of:

(a) contacting said biological sample with a magnetically-labelled ligand having selective binding affinity for the target bioentity, or for a determinant on the target bioentity, to form a target bioentity/labelled ligand complex in said biological sample;

(b) locating a separation module having a defined fluid flow path in the magnetic field of a magnetic module, said defined fluid flow path comprising a plurality of tubular elements having a common inlet and a common outlet, and said magnetic module comprising an array of at least two magnets in which adjacent magnets in the array are aligned with opposing polarity;

(c) passing said biological sample through the separation module to subject the biological sample to the magnetic field while the sample is passing through the defined fluid flow path to magnetically capture the target bioentity/labelled ligand complex by arresting or hindering movement of said complex within the defined fluid flow path;

(d) optionally, and preferably, passing a wash solution through the defined fluid flow path while said complex is magnetically captured within the fluid flow path;

(e) removing the separation module from the magnetic field; and

(f) recovering said target bioentity/labelled ligand complex from the fluid flow path.

The term “target bioentity” as used herein refers to a wide variety of materials of biological or medical interest. Examples include hormones, proteins, peptides, carbohydrates, lectins, oligonucleotides, drugs, chemical substances, nucleic acid molecules, (e.g., RNA and/or DNA) and particulate analytes of biological origin, which include bioparticles such as cells, viruses, bacteria and the like. In a preferred embodiment of the invention, rare or very rare cells, such as fetal cells in maternal circulation circulating tumour cells or circulating stem cells, may be efficiently isolated from non-target cells and/or other bioentities in a biological sample, using the method and apparatus of the present invention. The target bioentity may also comprise sub-cellular components, such as nucleic acids and exosomes, including exosomal contents such as proteins, carbohydrates and nucleic acids, particularly RNA, in a blood, plasma or serum sample.

The term “biological sample” includes, without limitation, cell-containing bodily fluids, peripheral blood, blood plasma or serum, saliva, tissue homogenates, lung and other organ aspirates, and lavage and enema solutions, and any other source that is obtainable from a human or animal subject. It will be appreciated that the method of the present invention is not restricted to the medical or veterinary fields, and may be applied widely to isolate a target bioentity from a biological sample in other fields, including non-medical applications such as food testing, agricultural testing, waste water testing and the like.

The term “determinant”, when used in reference to any of the foregoing target bioentities, may be specifically bound by a biospecific ligand, and refers to that portion of the target bioentity involved in, and responsible for, selective binding to a specific binding substance, the presence of which is required for selective binding to occur. In fundamental terms, determinants are molecular contact regions on target bioentities that are recognized by receptors in specific binding pair reactions.

The terms “specific binding” or “selective binding” as used herein include antigen-antibody, receptor-hormone, receptor-ligand, agonist-antagonist, lectin-carbohydrate, nucleic acid (RNA or DNA) or aptamer hybridizing sequences, Fc receptor or mouse IgG-protein A, avidin-biotin, streptavidin-biotin and virus-receptor interactions. Various other determinant-specific binding substance combinations are contemplated for use in practicing the methods of this invention, such as will be apparent to those skilled in the art. The term “antibody” as used herein, includes immunoglobulins, monoclonal or polyclonal antibodies, immunoreactive immunoglobulin fragments, and single chain antibodies. Also contemplated for use in the invention are peptides, oligonucleotides or a combination thereof which specifically recognize determinants with specificity similar to traditionally generated antibodies.

In accordance with the method of the present invention, a ligand having selective binding affinity for the target bioentity is magnetically-labelled, for example by coupling the ligand to suitable magnetic particles or beads. Magnetic particles are well known in the art, as is their use in immune and other bio-specific affinity reactions (see for example Whitehead et al., U.S. Pat. No. 4,554,088 and Terstappen et al., U.S. Pat. No. 7,332,288). These particles can be classified on the basis of their size as large (1.5 to about 50 microns) or small (0.7 to 1.5 microns).

Small magnetic particles are quite useful in analyses involving bio-specific affinity reaction, as they are conveniently coated with biofunctional polymers (e.g., proteins), provide very high surface areas and give reasonable reaction kinetics. Magnetic particles ranging from 0.7-1.5 microns have been described in the patent literature, including, by way of example, U.S. Pat. Nos. 3,970,518; 4,018,886; 4,230,685; 4,267,234; 4,452,773; 4,554,088; and 4,659,678.

Small magnetic particles, such as those mentioned above, generally fall into two broad categories. The first category includes particles that are permanently magnetizable, or ferromagnetic; and the second comprises particles that exhibit bulk magnetic behaviour only when subjected to a magnetic field. The latter are referred to as magnetically responsive particles. Materials displaying magnetically responsive behaviour are sometimes described as superparamagnetic. However, materials normally considered ferro-magnetic, e.g., magnetic iron oxide, may be characterized as superparamagnetic when provided in crystals of about 30 nm or less in diameter. Like the small magnetic particles mentioned above, large magnetic particles (>1.5 microns to about 50 microns) can also exhibit superparamagnetic behaviour. Typical of such materials are those described in U.S. Pat. No. 4,654,267 and manufactured by Dynal, (Oslo, Norway). The process involves the synthesis of polymer particles which are caused to swell and magnetite crystals are embedded in the swelled particles. Other materials in the same size range are prepared by synthesizing the polymer particles in the presence of dispersed magnetite crystals. This results in the trapping of magnetite crystals in a polymer matrix, thus making the resultant materials magnetic. In both cases, the resultant particles have superparamagnetic behaviour, which is manifested by the ability to disperse readily upon removal of the magnetic field. These materials, as well as small magnetic particles, are readily separated with simple laboratory magnetics because of the mass of magnetic material per particle.

When the magnetically-labelled ligand is contacted with the biological sample, the ligand selectively binds to the target bioentity, or to the determinant on the target bioentity (such as a tumour cell-specific biomarker), to form a target bioentity/labelled ligand complex coupled to the magnetic particles or beads within the biological sample.

If desired, more than one magnetically-labelled ligand may be used in order to enhance the sensitivity of the method. By way of example, to separate circulating tumor cells (CTCs) from a blood sample, magnetic particles labelled with anti-EpCAM (Epithelial Cell Adhesion Molecule) antibody may be used, however as EpCAM is not expressed on all epithelial cells and may only be weakly expressed on others, magnetically labelled anti-cytokeratin (CK) antibody may also be used either alone or in combination with magnetically-labelled anti-EpCAM antibody.

The biological sample is then passed through the separation module having a defined fluid flow path, which in one aspect may be a restricted fluid flow path such as a coiled tube or other configuration providing a suitable labyrinthine or tortuous fluid flow path, having a proximal (inlet) end and a distal (outlet) end, which is or can be located within the magnetic field of a magnetic module. In one embodiment, a tubular fluid flow path is formed from inert flexible plastic tubing such as PTFE (Teflon), PVC or silicone tubing. As an alternative to a tubular fluid flow path, a tortuous fluid flow path could also be achieved, for example, by providing fluid flow around a series of parallel plates within a container, or by molding or vacuum forming a fluid flow path into or onto a body of plastics material and if necessary sealing the path with an adhesive cover. Preferably, the defined fluid flow path is formed from inert, flexible plastic tubing having an internal diameter (ID) of 0.5 mm to 5 mm, preferably an ID of 0.8 mm to 1.6 mm, and a tube length (within the magnetic field) of 50 mm to 200 mm, preferably 100 mm to 125 mm.

The defined fluid flow path provided by the separation module may also consist of multiple flexible tubes, for example, multiple parallel flexible tubes, with the tubes converging to a common inlet and common outlet. Alternatively, the fluid flow path may be molded with parallel tracks rather than constructed of individual tubes. Both ends of the flow path are provided with means for supply of liquids to or from the flow path. In this aspect, the separation module may consist of 10 parallel silicon rubber tubes, 1 mm in diameter and 250 mm long arranged on a flat base, but gathered at each end into a bundle suitable for connection to fluid inlet and outlet means. Alternatively, the tubes may be connected to an inlet manifold at one end and an outlet manifold at the other end. Provision may be made for equal liquid flow through each tube, for example by the use of rollers such as used in peristaltic pumps.

In this aspect of the invention, the use of multiple tubular elements, particularly multiple parallel flexible tubes, may be particularly advantageous since it allows the flow of a larger sample volume (and therefore more target bioentity) through the defined fluid flow path at a lower flow rate (and hence with less shear forces) than if a single tubular element or tube is used.

As the biological sample is passed through the defined fluid flow path provided by the separation module from the proximal end to the distal end, it is subjected to the magnetic field of the magnetic module so that the target bioentity/labelled ligand complex is magnetically captured by arresting or hindering movement of the complex and holding it within the defined fluid flow path.

In one embodiment, a series of magnetic fields may be applied progressively along the defined fluid flow path, for example from the distal end to the proximal end, so that the magnetically-labelled complex is more uniformly captured along the fluid flow path rather than at just one point.

It has been noted that magnetic (or paramagnetic) particles flowing in a fluid-conducting tube across an array of magnets are primarily arrested at the junctions between magnets in the array. The present invention therefore provides a magnetic module including multiple magnets, or points for magnetic capture, in the fluid flow path to provide for maximum capture of particles. The magnetic module preferably employs permanent magnets to capture, concentrate, purify and release the magnetic particles, as follows:
a) a stationary permanent bar magnet array of, for example, 4 to 20 individual magnets may be used and provides a simple and effective magnetic module when the adjacent magnets in the array are aligned with opposing magnetic polarity;
b) arrays of multiple bar magnets may be arranged to rotate around, against or with the liquid flow to capture or release the magnetic particles.

In another embodiment, the separation module may also be located between two belts of moving magnet arrays which can be rotated so as to sweep the magnetic particles in the defined fluid flow path within the separation module in either direction.

The preferred magnetic module is an array of at least two rare earth permanent magnets, for example of approximately 50×5×3.5 mm, with the poles being on the two sides of 50×5 mm. It has been found the maximum arrest of flowing magnetic particles occurs at the junction between the magnets when they are aligned side-by-side and/or end-to-end with their poles up or down and alternating such that a magnet with its N pole up is next to one with its S pole up. To maximize particle capture, multiple magnets may be arranged in arrays consisting of linear pairs, blocks, rafts, on discs or around drums. In all cases, the fluid flow path of the separation module is arranged to allow maximum passage through or along the junctions between the magnets, i.e. the zone of maximum field intensity. Because the attractive forces are aligned, as a result of the opposing magnetic polarity of adjacent magnets in the array, the magnetic module has an integrity that requires only a simple support that provides ready access for the placement of the separation module. If required, this magnetic configuration also allows the magnets be placed so that they can be moved against the liquid flow to enhance capture, or with the flow to enhance particle release.

Since the points of maximum collection of magnetic particles are at or adjacent the N-S junctions between the magnets, not over the face of the magnets, the magnets may be configured in order to take advantage of this. For example, the magnets may be arranged:

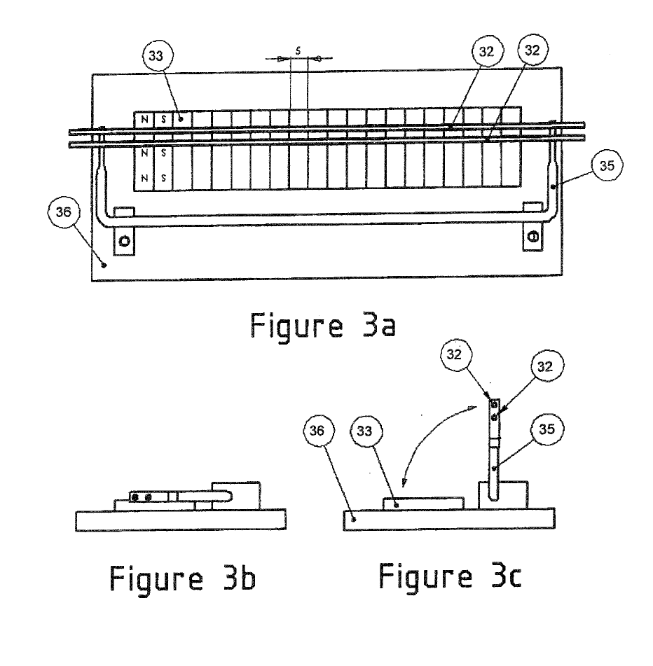

(a) In a “ladder”-like raft, with the tube(s) run perpendicular to the long axis of the individual magnets (as shown in FIG. 3 hereinafter). In this case there is discontinuous collection of particles at multiple arrest points, i.e. at the junctions.

(b) In parallel tracks, where the tube(s) run along the junctions between the long axes of the magnets. In this case there is continuous collection along the length of the tube/magnets.

The path required for the visible arrest of all magnetic particles is similar for both configurations (a) and (b), therefore the (a) configuration is generally preferable as more tubes may be run in parallel across the width of the magnetic module than with the limited number of magnetic junctions of configuration (b).

For example, in both cases (a) and (b), the magnetic module may consist of 20 magnets and be 50 mm wide and 100 mm long. For configuration (a) the only limit to the number of parallel tubes, and hence samples, that can be run across the magnetic module is that set by the tube width and its associated hardware. In contrast, in configuration (b) there are only 4 junctions between the magnets, so only 4 tubes can be used (or 4 samples processed) in parallel.

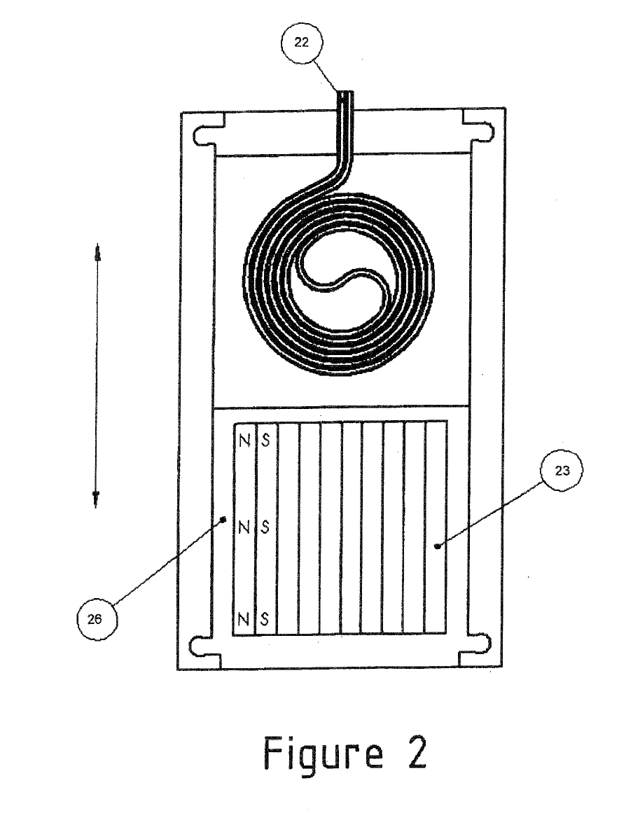

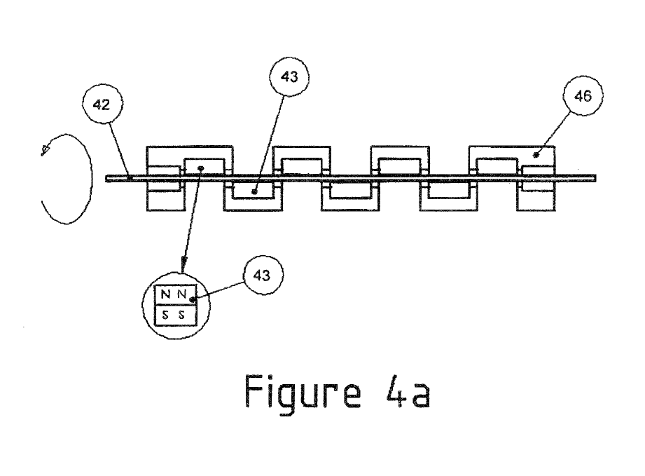

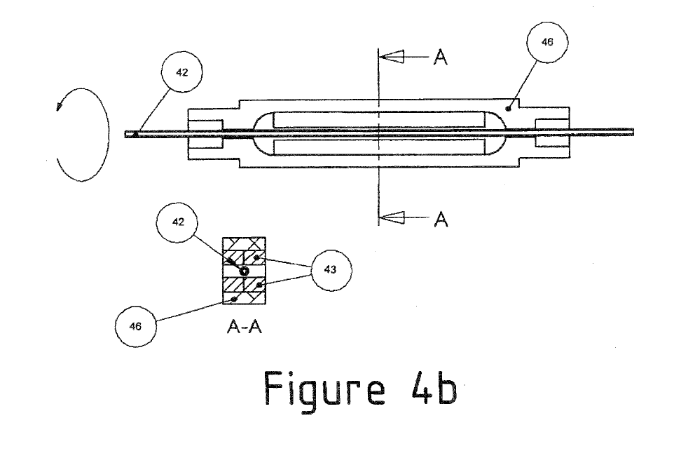

A coiled tube separation module (as shown in FIG. 2 hereinafter) is more compact than the linear models above and the particles are arrested at all junctions, lengthwise, crosswise and obliquely. A possible disadvantage is that it is not as suitable for multiple tubes (i.e. samples) as the linear models. A spinning magnet configuration (as shown in FIG. 4a hereinafter) enhances capture by passing the entities to be captured through a “cloud” of magnetic particles “hovering” in the bore of the tube. In static magnetic fields the magnetic particles stream to the side of the tube nearest the magnets and further arrest of the analyte is unlikely.

Use of spinning magnets, recirculation of the sample/particle mix through the separation module, and incubation of the mix in transit before separation are all aspects of the invention aimed at reducing or eliminating the reagent reaction-time requirement and bringing the testing closer to the physician or patient.

Preferably, after capture, the target bioentity/labelled ligand complex held within the defined fluid flow path is washed with a wash solution, preferably by repeated washing, to remove unbound cellular and other components of the biological sample. Preferably also, one or more air bubbles are fed along the fluid flow path to partition the biological sample from the wash liquid, and where repeated washing is performed to partition separate aliquots of wash liquid. By way of example, the order of fluids passed along the fluid flow path may be sample-air bubble-wash liquid-air bubble-wash liquid.

Finally, the captured complex is recovered from the defined fluid flow path by removing the separation module from the magnetic field of the magnetic module to release the magnetically-labelled complex, and then transferring the complex into a collection vessel, for example by passing a collection fluid through the fluid flow path. The isolated target bioentity/labelled ligand complex may then be concentrated by removal of the collection fluid if desired, and the complex is then suitable for further processing, for example, for analysis of biomarkers of disease.

In one embodiment, recovery of the captured complex may be achieved after the separation module has been removed from the magnetic field by back-flushing wash liquid from the distal or outlet end of the fluid flow path to flush the captured complex out the proximal or inlet end. Again, air bubbles may also be introduced between separate aliquots of wash liquid to assist in displacement and subsequent recovery of the captured complex.

Since the captured complex which is recovered from the separation module may also include unbound labelled ligand, for example magnetic particles which are not bound to target bioentities such as circulating tumour cells, the recovered material may be further treated to separate this unbound labelled ligand from the captured target bioentity/labelled ligand complex. This separation may be physical, for example, by use of a suitable membrane to separate the larger target bioentity/labelled ligand complex from the smaller unbound labelled ligand, or alternatively this separation may be achieved by use of a second binding agent to capture the target bioentity/labelled ligand complex and immobilise it on a solid support, for example, where the target bioentity is circulating tumour cells, the second binding agent may be anti-EpCAM antibody or anti-cytokeratin (CK) antibody coupled to biotin which binds to the target bioentity/labelled ligand complex (and not to the unbound labelled ligand) and the resulting complex can then be separated, for example, using streptavidin coupled to a tube or other solid support such as a microscope slide.

A suitable, programmed controller may be used to control flow of biological sample and wash liquids through the fluid flow path, and to introduce air bubbles as described above, as well as to control all other functions including, for example, movement of the separation module from the magnetic field, and recovery of the captured complex.

The further processing of the isolated target bioentity may include, for example, identification and assaying of isolated cells either directly using a disclosing reagent such as a fluorophore-labelled antibody and a fluorescence detector, or indirectly by examination of isolated cells such as by staining and/or microscopy, or by analysis for protein or nucleic acid biomarkers. In addition to these well known, traditional detection methods and assays, further processing of the isolated target bioentity may also include newer detection methodologies, such as assays based on epigenetic change, for example, changes to methylation status in methylation-based assays for prostate cancer.

As described above, the method of the present invention is suitable for the isolation of a wide range of target bioentities from biological samples, and requires only a suitable specific binding ligand which specifically binds to the target bioentity, or to a determinant on the target bioentity, and which can be coupled to magnetic particles or beads as described herein. The specific binding ligand may be an antibody, nucleic acid, or possibly even a chelating agent with an affinity for a particular element. The invention may therefore be useful for capture and concentration of any cells, including microorganisms, sub-cellular membrane bound vesicles, sub-cellular proteins and nucleic acids (including viruses), contaminants and toxins and elements such as rare earth elements.

In one particular embodiment, the method of the present invention is particularly suited for isolating a purified population of cells from a complex cellular medium such as blood. In this embodiment, the method includes the steps of:

(i) contacting the cellular medium (e.g. blood) with magnetic particles (e.g. 4.5 micron Dynabeads) which are coupled to a ligand which selectively binds to the population of cells of interest;

(ii) passing the cellular medium/labelled ligand mixture through a separation module having a defined fluid flow path which is located within the magnetic field of a magnetic module as described above to create a magnetic field around the fluid flow path in the separation module so that magnetic particles (and cells of interest bound thereto) in the mixture are captured and held within the separation module;

(iii) washing the captured magnetic particles (and cells of interest bound thereto) with a wash solution that removes unbound blood components;

(iv) removing the separation module from the magnetic field to release the magnetic particles; and

(v) recovering the particles, for example by elution using a flow of collection fluid through the tubular fluid flow path to transfer the magnetic particles (and cells of interest bound thereto) into a collection vessel for further processing and analysis.

In the method of this invention, the target bioentity/labelled ligand complex is subjected to a magnetic field as the biological sample is passed through the defined fluid flow path. In an alternative embodiment, the magnetic field may be pulsed or moved along the fluid flow path, or the biological sample subjected to equal and opposite magnetic forces, as the sample is passed through the defined fluid flow path. This alternative embodiment offers the potential of retaining the magnetic particles midstream of the fluid flow path rather than on the walls thereof, thereby enabling enhanced separation of the “bound” or captured magnetic particles from unbound components of the biological sample.

In one embodiment, the method of this invention may include the following steps:

1. Magnetic particles capable of acting directly or indirectly (e.g. via a biotin-streptavidin linkage) with the target entity are mixed with the fluid sample suspected of containing the entity.

2. After allowing time for reaction between the particles and the target entity, the fluid sample is drawn through the separation module at a rate that allows magnetic capture without loss of the target entity through the shear forces generated by the flow. By way of example, a syringe or similar device may be used at the downstream end of the separation module to draw the sample through the module.

3. After sample flow is completed, wash liquid (e.g. an isotonic buffer for cells) is flowed through the module to wash out any non-specifically bound materials.

4. After washing, and with the wash liquid still flowing, the separation module is removed from the magnetic field so that the magnetic particles are released and flowed from the separation module into a suitable collection vessel. Alternatively, the flow through the separation module may be reversed and the purified target entity expelled into a collection vessel at the proximal end the module.

Another embodiment of the method of the present invention addresses a limitation of the known prior art systems involving batch treatment to separate a target bioentity from a biological sample, for example to separate circulating tumour cells (CTCs) from a blood sample, which require time to be allowed for the passive reaction between any target bioentity (such as CTCs) in the sample and the magnetically-labelled ligand. In the method of the present invention, reaction of the target bioentity and magnetically-labelled ligand is assisted as the sample flows through the defined fluid flow path, but may be further enhanced by actively bringing the sample and magnetically-labelled ligand together within a sample tube, such as a Vacutainer blood collection vessel, rather than simply contacting the reagents and allowing time for passive, diffusion-limited reaction. By way of example, the magnetically-labelled ligand can be added to the sample (e.g. blood) in a sample tube which is situated over a magnet which would attract the magnetic particles through the sample to the base of the tube. In order to further enhance the reaction of the target bioentity and magnetically-labelled ligand, the sample can then be recirculated within the sample tube by drawing it from the base of the tube, i.e. through a high concentration of magnetic particles, and then added back to the top of the tube where the magnetic particles would again be attracted by the magnet through the sample to the base of the tube, and so on. In this recirculation process, CTC labeling, for instance, would thus be facilitated both by the constant passage of magnetic particles through the blood sample in the sample tube, and by the sample being drawn through a high concentration of magnetic particles in the base of the sample tube. At the completion of this active or accelerated labeling phase, the sample can be drawn from the base of the sample tube and on to the separation module to be passed through the defined fluid flow path as described above.

As described above, in another aspect, the present invention also provides an apparatus for use in isolating a target bioentity from a biological sample which comprises:

(i) a separation module having a defined fluid flow path;

(ii) a magnetic module having a magnetic field, said magnetic module comprising an array of at least two magnets in which adjacent magnets in the array are aligned with opposing magnetic polarity,

wherein the separation module has a first position in which the fluid flow path is located within the magnetic field of the magnetic module and a second position in which the fluid flow path is removed from said magnetic field; and

(iii) a controller comprising means for passing a biological sample, and optionally a wash solution, through the defined fluid flow path provided by the separation module whereby the biological sample is subjected to the magnetic field of the magnetic module while the sample is passing through the defined fluid flow path.

In one aspect, the present invention also provides an apparatus for use in isolating a target bioentity from a biological sample which comprises:

(i) a separation module having a defined fluid flow path, said defined fluid flow path comprising a plurality of tubular elements having a common inlet and a common outlet;

(ii) a magnetic module having a magnetic field, said magnetic module comprising an array of at least two magnets in which adjacent magnets in the array are aligned with opposing magnetic polarity,

wherein the separation module has a first position in which the fluid flow path is located within the magnetic field of the magnetic module and a second position in which the fluid flow path is removed from said magnetic field; and

(iii) a controller comprising means for passing a biological sample, and optionally a wash solution, through the defined fluid flow path provided by the separation module whereby the biological sample is subjected to the magnetic field of the magnetic module while the sample is passing through the defined fluid flow path.

As described above, in the apparatus of the invention the separation module has a first position in which the fluid flow path is located within the magnetic field of the magnetic module and a second position in which the fluid flow path is removed from the magnetic field. By way of example, the separation module may be moved in relation to a fixed or stationary magnetic module so that it moves from the first to the second position. Alternatively, the magnetic module may be moved in relation to a fixed or stationary separation module to achieve the same effect.

The method and apparatus of the invention is particularly suitable for commercial, automated operation. The separation module which includes the defined fluid flow path may be inexpensive and disposable, and capable of high-speed automated manufacture. Further, control of magnet activation and all other processes (such as aspiration of the biological sample through the separation module, passage of wash solution, and elution to recover the target bioentity) can be achieved automatically using suitable control instrumentation.

As outlined above, the method and apparatus of the invention are designed for detecting, concentrating and purifying target bioentities of interest including, but not limited to, extremely rare target bioentities from fluids. One preferred application is for detecting, purifying and concentrating circulating tumor cells (CTCs), or their nucleic acids, from blood. CTCs may be present in the blood of cancer patients at a concentration of less than one in one billion cells, but their presence and number is important for prognosis and managing the therapy of the patient.

The most widely used method for detecting CTCs is currently the Veridex CellSearch system which uses CTC detection for predicting survival and monitoring therapeutic response in cancer patients. The CellSearch method employs batch treatment of blood samples, and has the disadvantages of being slow and its effectiveness is limited by insufficient sensitivity. The background to this technology and the deficiencies of the current methods are disclosed by Talasaz et al (2009) Proc Natl Acad Sci USA 106:3970-3975, which also introduces an alternate, more sensitive approach to CTC collection. The present invention addresses the following deficiencies of both the CellSearch and Talasaz et al approaches, and provides a scalable means for CTC detection, purification and recovery suitable for high throughput diagnostic laboratory applications:

Sensitivity: The speed and sensitivity of the CellSearch method is restricted by the passive, diffusion-limited time for reaction between the magnetic particles and any CTCs in the blood. The present invention enhances capture by flowing CTCs in close proximity with the magnetic particles and this may be further promoted by incubation in transit and/or sweeping a “cloud” of “bounced” magnetic particles against the flowing blood into the upstream portion of the separation module so that the module acts like an affinity purification column. The ability to process large volumes of blood at low flow rates and low shear forces resulting from the use of multiple parallel tubular elements may also be used to increase the number of cells captured.

Purity: The CellSearch method traps debris in the magnetic deposits thereby requiring multiple wash steps with repeated deposition and re-suspension of the CTCs. This is time consuming and may be destructive of the cells. The present invention gently arrests CTCs in the separation module while extraneous materials flow to waste. Releasing and re-applying the magnetic field or “bouncing” the particles may further assist the release of any non-specifically bound materials.

Sample integrity: The CellSearch method provides CTC detection and cell counts, but does not provide cells for analysis beyond microscopic examination. The present invention provides a gentle, minimally destructive method for detecting, purifying and concentrating cells for further analysis, including protein and nucleic acid analysis.

Commercial application: The CellSearch method is capable of batch processing seven samples and one control in 3-4 hours. The Talasaz et al method may batch process more samples but the volume of sample, and hence number of CTCs, is limited and the magnetic sweeping and recovery method is unsuitable for high-throughput commercial applications. The present invention enables large volumes to be processed with efficient cell recovery and is scalable for commercial and laboratory applications.

While particularly suitable for CTC purification, the present invention is equally suitable for all applications that require recovery of minute quantities of a target entity from a large sample volume.

In a particularly preferred embodiment of this invention, the magnetic module comprises an array of multiple rare earth magnets, for example, 10 magnets of approximately 50×5×3.5 mm, with the poles being on the two 50×5 mm sides, forming an array or “mat” which is approximately 50×50 mm. The magnets are arranged side-by-side in this array with their poles up or down and with adjacent magnets having opposing magnetic polarity, that is, such that a magnet with its N pole up is adjacent to one with its S pole up. The array of magnets is mounted on a suitable base for the magnetic module.

In this embodiment, which has been adopted for suitability for nucleic acid capture and release as well as for capturing CTCs, in order to avoid the risk of contamination all components, including reagents, are provided in disposable for “single-use” form. These disposable components include

(i) A syringe (1−30 mL, preferably 10 mL)
(ii) A separation module which includes or consists of a hollow tube, similar to an elongated disposable pipette tip, and may be in a straight, coiled or serpentine configuration. The proximal end of the module has a female taper to allow a sealing fit with a laboratory pipettor or a syringe. The distal end has a male taper with an outlet ID similar to that of a disposable pipette tip. The appropriate tube bore diameter (ID) would be selected for the chosen application, most likely between 0.5 mm and 5 mm and with a tube length between 50 and 200 mm. The preferred ID is 1 mm, with a tube length of 100 to 125 mm. The separation module material may be of any rigid or semi-rigid inert material including a variety of plastics, elastomers, glass or metal. Less rigid materials e.g. PTFE (Teflon) or silicon rubber tubing may also be used if a rigid or semi-rigid support is provided for the module.
(iii) A waste reservoir for receipt of the effluent from the separation module.
(iv) Reagent containers or reservoirs for provision of wash and other reagents e.g. for labeling or lysing cells. The reagents may be provided in bottles or wells in a disposable tray, e.g. with seals that may be penetrated by the distal (male) end of the module. The tray may also house the waste reservoir.

While magnetic separations may be performed manually with the magnetic module described above and the above disposable components, an automated or semi-automated instrument is clearly preferable for control, safety and containment. Such an instrument would include the following:

(v) A syringe driver, i.e. a reversible drive for expelling liquids from, or drawing them into, the syringe.
(vi) A means for supporting and holding the separation module in close proximity to the magnetic module consisting of multiple strong rare earth magnets as previously described.
(vii) A waste reservoir or means for receiving waste effluent from the separation module.
(viii) A means for providing reagents to the distal end of the separation module. For example, this may be a slidable or rotary mechanism that presents the reagent containers to the distal end of the separation module.
(ix) A programmable controller to control the functions of the instrument.

The following describes, by way of example, a method for isolation of circulating tumor cells (CTCs) from blood by use of magnetic particles labelled with anti-EpCAM (Epithelial Cell Adhesion Molecule) antibody, but is equally suitable for other magnetic separations, including purification of nucleic acids. The description assumes that the waste and reagent vessels are provided in a rotary or movable rack or tray that is capable of presenting them to the separation module.

a. Draw the required volume (typically 7.5 mL) of blood into the syringe. Preferably the blood will be supplied in a Vacutainer containing anti-EpCAM magnetic particles, or alternatively, the magnetic particles may be issued in, or added to, the syringe.
b. Couple the syringe and separation module, locate the module in the magnetic field of the magnetic module and engage the syringe plunger in the syringe driver.
c. Start the syringe driver:
a. Blood passes through the separation module at a controlled rate and is received in the waste reservoir. (During this step, which typically takes 5 minutes, magnetic particles and bound CTCs are arrested in the separation module).
b. The waste reservoir is moved away and wash liquid is drawn from its reservoir through the separation module into the syringe. This leaves washed CTCs in the separation module. If required, lysing or staining reagents may also be drawn through the separation module
   d. Remove and discard the syringe.
   e. Insert a pipettor into the separation module (which becomes the pipette tip) and remove the separation module from the magnetic field.
   f. Expel the separation module contents (CTCs and magnetic particles) into a microtiter well (or similar). If necessary, additional recovery may be obtained by repeatedly drawing the sample up and down through the separation module. If required, a further wash step could also be used. Using this apparatus, it has been found that a separation module which includes a tube of the preferred dimensions (ID=1 mm, L=100 mm) recovers the CTCs in a volume of ˜80 uL of liquid. (If L=125 mm the volume is ˜100 uL).

The above method and apparatus has a number of advantages:
It is exceedingly simple and inexpensive as it has no valves or plumbing.
It is suitable for molecular biology, infectious agent and enzymic separations as all components and reagents are disposable, thereby eliminating any risk of contamination.
All disposables are either readily available or easy and inexpensive to manufacture.
Separations may be multiplexed. For example, separation modules may be made in joined sets of 8 or 12 (individually separable), spaced so as to be suitable for use with standard laboratory pipettors and microtiter trays.

In an alternative method to the method for isolating CTCs from a blood sample as described above, the blood sample is supplied in a Vacutainer collection vessel containing anti-EpCAM magnetic particles, the syringe is coupled to the separation module (which is located in the magnetic field of the magnetic module) and the blood sample is drawn from the collection vessel through the separation module and into the syringe so that magnetic particles and bound CTCs are arrested in the separation module. After the blood sample has been drawn from the collection vessel, an air bubble is drawn into the separation module followed by wash liquid which is drawn from its reservoir and into the syringe, leaving the washed CTCs in the separation module. The separation module is then removed from the magnetic field of the magnetic module, and the CTCs are recovered from the separation module as described above.

A number of variations or modifications may be made to the apparatus as described above. For example, the apparatus may be modified in order to provide multi-stage capture of the desired analyte. Thus, in order to improve the purity of captured CTCs, a two-stage approach using two separation modules in series may be used. Magnetic particles are bound CTCs are captured in the first separation module and washed as described above, the first separation module is removed from the magnetic field of the magnetic molecule, then the second separation module is located in the magnetic field and the magnetic particles and bound CTCs from the first separation module are passed to and captured in the second separation module before being recovered as described above.

Similarly, multi-stage capture may also be used for extraction of RNA and/or exosomes from cancer cells such as CTCs. In this embodiment, a first separation module is used as broadly described above in order to capture CTCs (bound to magnetic particles) from a blood sample. The captured and washed cells from the first separation module are lysed in the first separation module so that cellular debris bound to the magnetic particles remains captured in that separation module and lysed material (including DNA and/or exosomal contents) is recovered into a collection vessel. The lysed material is mixed in the collection vessel with RNA-binding magnetic particles (e.g. magnetic glass beads) and, if desired, RNA extraction reagents, and then passed through a second separation module located in the magnetic field of the magnetic module where the magnetic particles and bound RNA are captured. These magnetic particles and bound RNA are then recovered from the second separation module by removing it from the magnetic field and recovering the captured material as previously described.

Multi-stage capture may also be used for removal of non-specifically captured cells such as lymphocytes from captured CTCs. In this embodiment, CTCs bound to magnetic particles are captured and washed as described above. The CTCs are recovered and released into a reaction vessel where they are mixed with an anti-lymphocyte antibody (such as anti-CD45) bound on large, non-magnetic beads to label lymphocytes. The mixture is then passed through a second separation module located in the magnetic field to again capture CTCs bound to magnetic particles, with lymphocytes bound to the large, non-magnetic beads being swept through this separation module. The captured CTCs are then recovered from the second separation module by removing it from the magnetic field, and recovering the captured material as previously described.

In another aspect, in order to speed up the labelling reaction and shorten processing times, the method of the invention may include the steps of:

collecting said biological sample at a first or collection point;

(ii) adding said sample at said first point to a collection medium comprising said magnetically-labelled ligand;

(iii) subsequently forwarding said collection medium comprising said sample and magnetically-labelled ligand to a second or testing point; and

(iv) completion of said test method at said second point.

The biological sample may be treated directly to isolate a target bioentity according to the method of the present invention as described above or may require some form of treatment prior to testing. For example, a biopsy sample may require homogenisation prior to testing. Further, to the extent that the biological sample is not in liquid form, (for example it may be a solid, semi-solid or dehydrated liquid sample) it may require the addition of a reagent, such as a buffer, to mobilize the sample.

The “collection medium” to which the biological sample is added in accordance with the present invention may be any suitable medium which is well known to persons skilled in the art. Suitable media include, by way of example, saline and buffered media suitable for the collection and transport of biological samples. The collection medium includes the magnetically-labelled ligand as described above, and may also contain stabilizing agents for the sample and ligand, before and after addition of the biological sample to the collection medium, including, for example, in addition to buffer, detergent(s), protease inhibitor(s) and stabilizers.

In a particular embodiment, the present invention provides a method as broadly described above wherein the collection medium is designed to enhance protein solubility for subsequent or simultaneous reaction to detect one or more analytes. Such reagents to enhance solubility of target peptides or in the sample are well known to persons skilled in the art and include, for example, surfactants, buffers of high or low ionic strength, anti-mucolytic agents, lysozymes, and nucleases. Furthermore, in this embodiment, the invention also includes the addition of specific proteases to selectively degrade target peptides or proteins to expose specific epitopes. In this embodiment of this invention, the inclusion of such solubility reagents in the collection medium assists in the detection of protein or peptide gene expression products in the sample since these reagents are active in enhancing the solubility of target expression products during the transit time of the sample from the first or collection point to the second or testing point.

The following examples of the use of the present invention for isolation of circulating cancer cells from a blood sample are described by way of illustration, and not limitation, of the invention.

### EXAMPLES

### Example 1

### Recovery of Latex Microspheres from Buffer

Biotin-labeled latex microspheres (Bangs Laboratories, diameter of 10.35 um, 10 ul) were mixed with Streptavidin coated magnetic particles (Chemicell, 1 um diameter, 10 ul) and reacted for 30 minutes at ambient temperature in 7.5 ml of Phosphate Buffered Saline (PBS, pH 7.2). The mix was drawn through the apparatus as illustrated in FIG. 2, comprised of a 1 mm×200 mm coiled PTFE tube (the separation module) over a magnetic module comprised of a raft 10 rare earth magnets (each 5 mm×3.5 mm×50 mm, arranged side by side with alternating polarity, i.e. a total dimension of 50 mm×50 mm×3.5 mm) so that flow was completed in approximately 5 minutes. After washing with 1 ml of PBS, the separation module was removed from the magnetic module and the captured particles back-flushed from the module with air bubbles and PBS into a small tube over a magnet. The supernatant liquid was removed and microscopic examination of the resuspended deposit showed that both the smaller magnetic particles and larger latex particles were captured in the separation module. Centrifugation of the effluent yielded no deposit, indicating that the magnetic particles had bound the larger latex particles and that all particles of both species were captured in the separation module.

### Example 2

### Recovery of Latex Microspheres from Blood

The experiment of example 1 was repeated with fresh human blood instead of PBS. A small air bubble was introduced after blood flow was completed and again after commencement of the wash to assist in purging residual blood from the separation module. Microscopic examination of the captured particles showed a dense mixture of latex beads and smaller magnetic particles.

### Example 3

### Recovery of Cultured Human Colorectal Cancer Cells from Blood

### (a) Coiled Tube Separation Module (FIG. 2)

Cultured human colorectal cancer cells (HCT 116, ATCC no. CCL-247) were harvested, counted and made to a known concentration of cells per ml using culture fluid as diluent. Cells were added to 5 ml aliquots of fresh human blood to a total of 1000, 500, 250, 50 and 8 cancer cells per aliquot. Magnetic beads coated in anti-epithelial cell antibody (Dynabead Epithelial Enrich, anti-EpCAM) were added (10 ul) to each aliquot and the mixtures incubated at room temperature for at least 30 minutes. Each blood mixture was run through the apparatus of FIG. 2 at a flow rate of approximately 1.67 ml/minute (i.e. 3 minutes flow time for 5 ml), followed by a wash of 1 ml of PBS containing 0.05% Tween 20 (PBST). The brown colored magnetic particles were observed to be arrested in the first half (100 mm) of the coiled tube. After removal from the magnetic module, the separation module was back-flushed with PBST (1 ml) and air bubbles into a small tube. The magnetic particles were concentrated over a magnet into the base of the tube, the supernatant removed and the particles resuspended in 10 ul of 25% methylene blue stain in PBST. In all cases microscopic examination of the recovered materials showed blue stained cells of the type added and magnetic particles, with the concentration of cells approximately equivalent to the number originally added. In the case of the blood with 8 added cells, one cluster of 6 cells and 2 single cells were found. In no case was any red or white blood cells observed.

### (b) Straight-Tube Separation Module (FIG. 3)

One hundred HCT 116 cells were added to 5 ml of blood and 10 ul of magnetic beads, incubated for 30 minutes at room temperature and drawn at approximately 1.67 ml/minute through a straight PTFE tube (length=200 mm, ID=1 mm) over a raft of 20 rare earth magnets as illustrated in FIG. 3 (i.e. Magnetic module=100×50×3.5 mm). Brown colored magnetic particles were arrested in the separation module tube within the first 75 mm of the Magnetic module length. As for example 3(a), after removal from the magnets the separation module was back-flushed, the particles recovered and found to be comprised of magnetic particles and cells resembling those added. No blood cells were visible.

### (c) Straight Separation Module Tube and Alternate Magnet Pairs: FIG. 4a

Magnet pairs were arranged in series in an alternating configuration (FIG. 4a) and rotated at 140 rpm around a straight PTFE tube (1 mm diameter, 200 mm long). When 5 ml of PBS containing 10 ul of magnetic particles was flowed through the tube at a rate of 1.67 ml/minute the first magnet pair arrested all particles. When the same volume (5 ml) of blood containing the same amount (10 ul) of magnetic particles was drawn through the tube at the same flow rate, nothing was captured. When the flow rate was reduced to 0.625 ml/min the particles were arrested in the tube within a total magnet length of approximately 60 mm. This suggested that the spinning magnets caused the particles to form a “cloud” in the centre of the tube that was displaced by the more dense blood at higher flow rates.

### (d) Straight Separation Module Tube and Spinning Opposite Magnet Pairs: FIG. 4b

In this example a reduced blood flow velocity was achieved by using a larger diameter tube in the Separation module and a slower flow rate. A straight PTFE tube of 1.6 mm diameter and length of 150 mm was threaded between two opposite magnet pairs, each of 50×5×3.5 mm, in a magnetic module as shown in FIG. 4b. The magnets were rotated at 140 rpm around the tube and 5 ml of blood containing 200 HCT 116 cells and 10 ul of magnetic particles passed through the module at a rate of 0.625 ml/minute. All particles were arrested in the first 25 mm of the length of the magnets. After a 1 ml wash with PBST the tube was withdrawn from the magnetic module and the captured particles recovered as previously described. The recovered material was found to be magnetic particles and cells resembling those added. No blood cells were visible.

1. A method for isolating a target bioentity from a biological sample, which comprises the steps of:
(a) contacting said biological sample with a magnetically-labelled ligand having selective binding affinity for the target bioentity, or for a determinant on the target bioentity, to form a target bioentity/labelled ligand complex in said biological sample;
(b) locating a separation module having a defined fluid flow path in the magnetic field of a magnetic module, said magnetic module comprising an array of at least two magnets in which adjacent magnets in the array are aligned with opposing polarity;
(c) passing said biological sample through the separation module to subject the biological sample to the magnetic field while the sample is passing through the defined fluid flow path to magnetically capture the target bioentity/labelled ligand complex by arresting or hindering movement of said complex within the defined fluid flow path;
(d) optionally, and preferably, passing a wash solution through the defined fluid flow path while said complex is magnetically captured within the fluid flow path;
(e) removing the separation module from the magnetic field; and
(f) recovering said target bioentity/labelled ligand complex from the fluid flow path.

2. A method for isolating a target bioentity from a biological sample, which comprises the steps of:
(a) contacting said biological sample with a magnetically-labelled ligand having selective binding affinity for the target bioentity, or for a determinant on the target bioentity, to form a target bioentity/labelled ligand complex in said biological sample;
(b) locating a separation module having a defined fluid flow path in the magnetic field of a magnetic module, said defined fluid flow path comprising a plurality of tubular elements having a common inlet and a common outlet, and said magnetic module comprising an array of at least two magnets in which adjacent magnets in the array are aligned with opposing polarity;
(c) passing said biological sample through the separation module to subject the biological sample to the magnetic field while the sample is passing through the defined fluid flow path to magnetically capture the target bioentity/labelled ligand complex by arresting or hindering movement of said complex within the defined fluid flow path;
(d) optionally, and preferably, passing a wash solution through the defined fluid flow path while said complex is magnetically captured within the fluid flow path;
(e) removing the separation module from the magnetic field; and
(f) recovering said target bioentity/labelled ligand complex from the fluid flow path.

3. The method according to claim 1 or claim 2, wherein the biological sample is a blood, plasma or serum sample.

4. The method according to claim 3, wherein the target bioentity is a nucleic acid, a protein or a carbohydrate.

5. The method according to claim 3, wherein the target bioentity is a rare or very rare circulating tumour cell, or a sub-cellular component thereof.

6. The method according to claim 1 or claim 2, wherein the biological sample is a maternal blood sample, and the target bioentity is a circulating fetal cell.

7. The method according to claim 1 or claim 2, wherein the magnetically-labelled ligand comprises a ligand having specific binding affinity for the target bioentity, or for a determinant on the target bioentity, coupled to magnetic particles or beads.

8. The method according to claim 7, wherein the ligand is an antibody, aptamer or nucleic acid.

9. The method according to claim 1 or claim 2, wherein in step (a) said biological sample is contacted with more than one magnetically-labelled ligand.

10. The method according to claim 1 or claim 2, wherein in step (a), one or more aliquots of wash solution are passed through the defined fluid flow path after said biological sample, and air bubbles are passed thorough the defined fluid flow path between the biological sample and the wash solution and optionally between aliquots of the wash solution.

11. The method according to claim 1 or claim 2, wherein recovery of the target bioentity/labelled ligand complex includes the step of flushing said complex from the defined fluid flow path by passing one or more aliquots of wash solution through the flow path, optionally with air bubbles between aliquots of the wash solution.

12. The method according to claim 11, wherein said flushing step comprises back-flushing through the defined fluid flow path.

13. The method according to claim 1 or claim 2, wherein recovery of the target bioentity/labelled ligand complex includes the further step of separation of said complex from unbound magnetically-labelled ligand.

14. The method according to claim 13, wherein said separation step is a physical separation step.

15. The method according to claim 13, wherein in said separation step a second binding agent is used to capture the target bioentity/labelled ligand complex and immobilise it on a solid support.

16. The method according to claim 1 or claim 2, wherein the target bioentity/labelled ligand complex recovered from the fluid flow path in step (f) is further purified by passing the recovered complex through a second separation module located in the magnetic field of a magnetic module, optionally washing the captured complex, and recovering the target bioentity/labelled ligand complex from the second separation module after it is removed from the magnetic field.

17. The method according to claim 1 or claim 2, wherein circulating tumor cells (CTCs) are isolated from a blood sample in a first separation module, captured and washed CTCs are lysed in the first separation module and lysed material from the CTCs is recovered from the first separation module, the recovered lysed material is contacted with a magnetically-labelled ligand for a target molecule and passed through a second separation module located in the magnetic field of a magnetic module to magnetically capture the magnetic particles and bound target molecule, and the magnetic particles and bound target molecule are then recovered from the second separation module after it is removed from the magnetic field.

18. The method according to claim 17 wherein the target molecule is a nucleic acid, a protein or a carbohydrate.

19. The method according to claim 1 or claim 2, wherein non-specifically captured cells such as lymphocytes are removed from CTCs isolated from a blood sample in a first separation module by contacting captured and washed magnetically-bound CTCs recovered from the first separation module with an anti-lymphocyte antibody bound on large non-magnetic beads to label lymphocytes, passing the mixture through a second separation module located in the magnetic field of a magnetic module to capture magnetically-bound CTCs and separate them from non-magnetically bound lymphocytes, and the magnetically-bound CTCs are then recovered from the second separation module after it is removed from the magnetic field.

20. The method according to claim 1 or claim 2, wherein in step (a) contact between said biological sample and said magnetically-labelled ligand is enhanced by (i) contacting the biological sample with the magnetically labelled ligand at the point of sample collection so that the target bioentity/labelled ligand complex forms in-transit between the collection site and the test site, or (ii) concentrating the magnetically-labelled ligand by means of a magnet placed beneath the base of a vessel containing the biological sample and ligand and recirculating the sample and ligand in the vessel, or (iii) forming a cloud of suspended magnetically-labelled ligand particles in the fluid flow path of the biological sample by means of magnets moving around or along the flow path.

21. An apparatus for use in isolating a target bioentity from a biological sample which comprises:
(i) a separation module having a defined fluid flow path;
(ii) a magnetic module having a magnetic field, said magnetic module comprising an array of at least two magnets in which adjacent magnets in the array are aligned with opposing magnetic polarity;
wherein the separation module has a first position in which the fluid flow path is located within the magnetic field of the magnetic module and a second position in which the fluid flow path is removed from said magnetic field; and
(iii) a controller comprising means for passing a biological sample, and optionally a wash solution, through the defined fluid flow path provided by the separation module whereby the biological sample is subjected to the magnetic field of the magnetic module while the sample is passing through the defined fluid flow path.

22. An apparatus for use in isolating a target bioentity from a biological sample which comprises:
(i) a separation module having a defined fluid flow path, said defined fluid flow path comprising a plurality of tubular elements having a common inlet and a common outlet;
(ii) a magnetic module having a magnetic field, said magnetic module comprising an array of at least two magnets in which adjacent magnets in the array are aligned with opposing magnetic polarity;
wherein the separation module has a first position in which the fluid flow path is located within the magnetic field of the magnetic module and a second position in which the fluid flow path is removed from said magnetic field; and
(iii) a controller comprising means for passing a biological sample, and optionally a wash solution, through the defined fluid flow path provided by the separation module whereby the biological sample is subjected to the magnetic field of the magnetic module while the sample is passing through the defined fluid flow path.

23. The apparatus according to claim 21 or claim 22, wherein the defined fluid flow path is formed from inert, flexible plastic tubing.

24. The apparatus according to claim 23, wherein the tubing has an internal diameter (ID) of 0.5 mm to 5 mm, preferably 0.8 mm to 1.6 mm, and a length of 50 mm to 200 mm, preferably 100 mm to 125 mm.

25. The apparatus according to claim 21 or claim 22, wherein the magnetic module comprises an array of at least two rare earth permanent magnets which are aligned side-by-side with adjacent magnets having opposing magnetic polarity.

26. The apparatus according to claim 21 or claim 22, wherein the separation module is movable with respect to the magnetic module which is fixed or stationary.

27. The apparatus according to claim 21 or claim 22, wherein the magnetic module is movable with respect to the separation module which is fixed or stationary.

28. A method according to claim 1 or claim 2, comprising the steps of:
(i) collecting said biological sample at a first or collection point;
(ii) adding said sample at said first point to a collection medium comprising said magnetically-labelled ligand;
(iii) subsequently forwarding said collection medium comprising said sample and magnetically-labelled ligand to a second or testing point; and
(iv) completion of said method at said second point.

29. The method according to claim 28, wherein the first and second points are separated, for example are remote or distant, from one another.

30. The method according to claim 28 or claim 29, wherein the steps of collection of the sample and addition of the sample to the collection medium on the one hand, and the step of completion of the method on the other hand, are separated in time from one another, in particular by a period of transit time sufficient not only to transport the collection medium from the first point to the second point, but also to enable formation of a target bioentity/labelled ligand complex while the sample is in transit.
